(19) United States
(12) Patent Application Publication
(10) Pub. No.: US 2004/0005669 A1
(43) Pub. Date: Jan. 8, 2004

(54) Recombinant expression of insulin C-peptide

(76) Inventors: Stefan Stahl, Stockholm (SE); Mathias Uhlen, Stockholm (SE); Per-Ake Nygren, Stockholm (SE); Per Jonasson, Stockholm (SE)

Correspondence Address: FISH & RICHARDSON PC, 225 FRANKLIN ST, BOSTON, MA, 02110, US

(21) Appl. No.: 10/430,752

(22) Filed: May 6, 2003

Related U.S. Application Data
(63) Continuation-in-part of application No. 09/485,286, filed on Feb. 7, 2000, now Pat. No. 6,558,924.
Application No. 09/485,286 is a 371 of international application No. PCT/GB98/02382, filed on Aug. 8, 1998.

(30) Foreign Application Priority Data
Aug. 7, 1997 (GB) 9716790.2

(51) Int. Cl. C12P 21/02; C12N 5/06; C07K 14/62; C07H 21/04

(52) U.S. Cl. 435/69.4; 435/320.1; 435/325; 530/303; 536/23.5

(57) ABSTRACT

The present invention provides a method of producing an insulin C-peptide, which comprises expressing in a host cell a multimeric polypeptide comprising multiple copies of a said insulin C-peptide, and cleaving said expressed polypeptide to release single copies of the insulin C-peptide.

Also provided are nucleic acid molecules, expression vectors and host cells, for use in such a method and the multimeric insulin C-peptide poolypeptide expressed and cleaved in such a method.

This application is a continuation-in-part of U.S. application Ser. No. 09/485,286, filed Feb. 7, 2000, which is the National Stage of International Application No. PCT/GB98/02382, filed Aug. 8, 1998, which claims foreign priority from British patent Application No. 9716790.2, filed Aug. 7, 1997, the contents of which are incorporated here by reference.

The present invention relates to the production of insulin C-peptide from recombinant DNA molecules comprising multimeric copies of a gene sequence encoding said insulin C-peptide.

Insulin is a protein hormone involved in the regulation of blood sugar levels. Insulin is produced in the liver as its precursor proinsulin, consisting of the B and A chains of insulin linked together via a connecting C-peptide (hereinafter this C-peptide derived from the proinsulin molecule is referred to as “insulin C-peptide”). Insulin itself is comprised of only the B and A chains. Several recent studies indicate that the C-peptide has a clinical relevance (Johansson et al., Diabetologia (1992) 35, 121-128 and J. Clin. Endocrinol. Metab. (1993) 77, 976-981). In patients with type 1 diabetes, who lack endogenous C-peptide, administration of the peptide improves renal function, stimulates muscle and glucose utilization and improves blood-retinal barrier function (Johansson et al., 1992 and 1993 supra).

Although not yet widely recognised, there is a growing awareness in the medical field of a therapeutic utility for the insulin C-peptide. Accordingly, there is a need for a method for the ready synthesis of insulin C-peptides, economically and efficiently. Whilst methods for the chemical synthesis of peptides, e.g. by stepwise addition of amino acids on a solid support, are now well developed, they remain, despite automation, time-consuming and, more significantly, costly to perform, and may also be limited in terms of the maximum peptide length economically and reliably synthesisable. As an alternative, methods for peptide production by expression of recombinant DNA have been developed, although these too are not without their drawbacks e.g. in terms of yield.

Current production schemes for insulin C-peptide are based on the processing of proinsulin, the precursor molecule for insulin and C-peptide, normally by the use of trypsin and carboxypeptidase B (Nilsson et al., (1996), J. Biotechnol. 48, 241-250); Jonasson et al., (1996) Eur. J. Biochem. 236, 656-661). Proinsulin was produced as a fusion protein that was capable of expression at high levels in E. coli, and the fusion protein was engineered in such a way that the fusion partner could be cleaved off simultaneously with the processing of proinsulin to insulin and C-peptide. Proinsulin was produced as a fusion protein with ZZ, a synthetic affinity fusion tag derived from staphylococcal protein A which binds IgG (Immuno-globulin) (Nilsson et al., (1987) Prot. Eng. 1, 107-113). This fusion tag was selected due to its stability to proteolysis, its IgG-binding capacity, its high expression levels and solubilizing properties. The chosen production strategy allowed the use of an affinity tag for efficient purification, after solubilization of inclusion bodies and subsequent renaturation, without the inclusion of additional unit operations for cleavage and removal of the ZZ affinity tag. The tag was demonstrated to be simultaneously cleaved off with the trypsin/carboxypeptidase B digestion of proinsulin to insulin and C-peptide. However, production of small peptides via the expression of large fusion proteins generally gives rather low yields, as the final product constitutes only a small part of the expressed gene product.

Shen in Proc. Natl. Acad. Sci. USA, 81, 4627-4631, 1984 describes a method for preparing human proinsulin by expression of a fused or unfused gene product comprising multiple tandemly linked copies of the proinsulin polypeptide domain. This gene product can be cleaved into single proinsulin units by cyanogen bromide treatment. It is proposed that human insulin can be prepared by cleavage of the proinsulin units with trypsin/carboxypeptidase. However, the problem of improving the yield of insulin C-peptide is not addressed.

There remains, therefore, a need for a recombinant expression method which improves the yield of insulin C-peptide, as an unfused product. The present invention addresses this need.

The present invention seeks to improve on existing methods for recombinant expression of peptides and essentially is based on the concept of increasing the amount of expressed target peptide (in this case an Insulin C-peptide) by expressing, as a single gene product, a multimer (i.e. a multimeric polypeptide) having multiple copies of the target peptide (insulin C-peptide), and then cleaving such a multimeric gene product (i.e. the multimeric polypeptide) to release the target peptide as individual monomer units.

In one aspect, the present invention thus provides a method of producing an insulin C-peptide, which comprises expressing in a host cell a multimeric polypeptide comprising multiple copies of a said insulin C-peptide, and cleaving said expressed polypeptide to release single copies of the insulin C-peptide (ie. to release the insulin C-peptide monomers from the multimer).

The multimeric polypeptide (gene product) is encoded by a genetic construct (in other words a nucleic acid molecule) comprising multiple copies of a nucleotide sequence encoding an insulin C-peptide. The multiple copies, or repeats, are linked in the construct in such a manner that they are transcribed and translated together into a single, multimeric gene product (i.e. a multimeric polypeptide) i.e. in “read-through format” e.g. the multiple nucleotide sequences are linked in matching reading frame in the construct. In essence, the genetic construct (nucleic acid molecule) advantageously comprises a concatemer of the insulin C-peptide encoding nucleotide sequence. Preferably, the genetic construct comprises tandem copies of the encoding nucleotide sequence. Such a genetic construct is thus prepared and is then introduced into a host cell in a standard manner, and expressed. The expressed gene product (polypeptide) may then be recovered and cleaved to release the insulin C-peptide monomers.

In a further aspect the invention thus provides a method for producing an insulin C-peptide, which comprises culturing a host cell containing a nucleic acid molecule comprising multiple copies of a nucleotide sequence encoding a said insulin C-peptide, under conditions whereby the multimeric polypeptide of said nucleic acid molecule is expressed, and cleaving said expressed polypeptide to release single copies of said insulin C-peptide.

As used herein the term “multiple” or “multimeric” refers to two or more copies of an insulin C-peptide or the nucleotide sequence which encodes it, preferably 2 to 50, 2 to 30 or 2 to 20, more preferably 2 to 15, or 2 to 10. Further exemplary ranges also include 3 to 20, 3 to 15 or 3 to 10.

Conveniently, the construct comprises 3 or more copies e.g. 3 to 7, or 5 to 7, copies of the nucleotide sequence encoding a insulin C-peptide. Ranges of 7 or more, for example 7 to 30, 7 to 20 or 7 to 15 may also be useful.

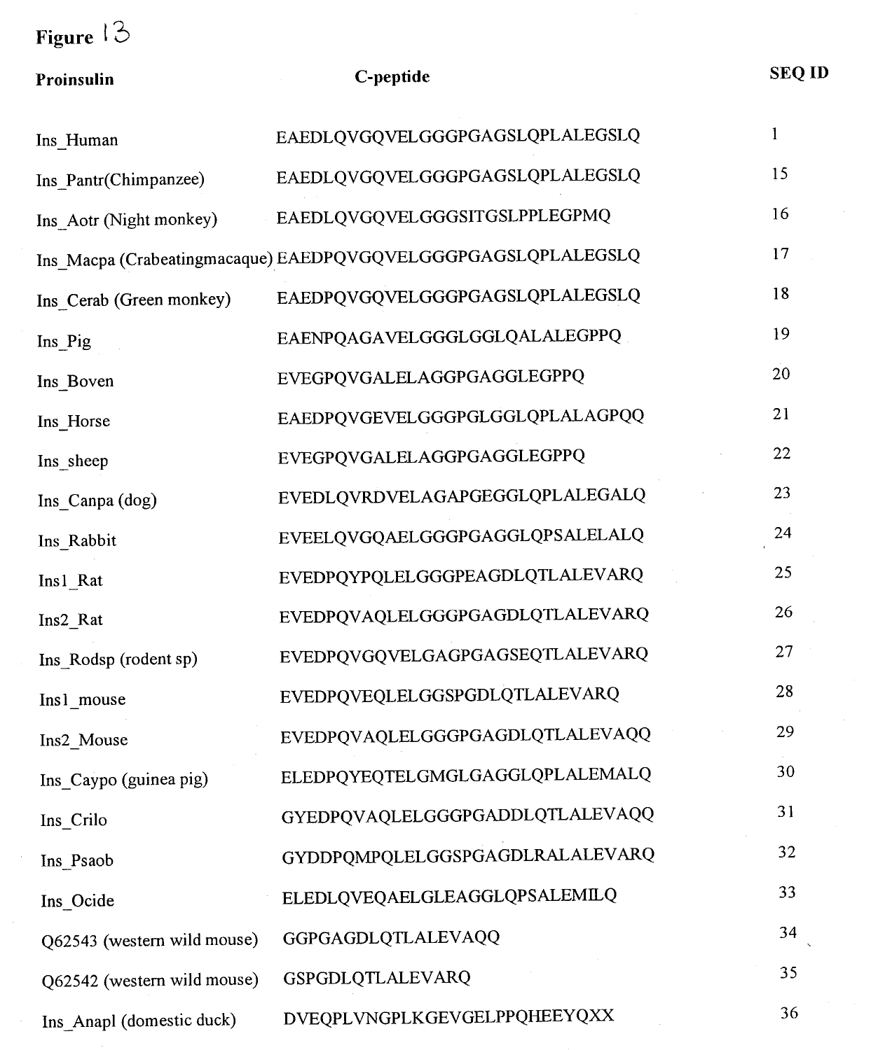

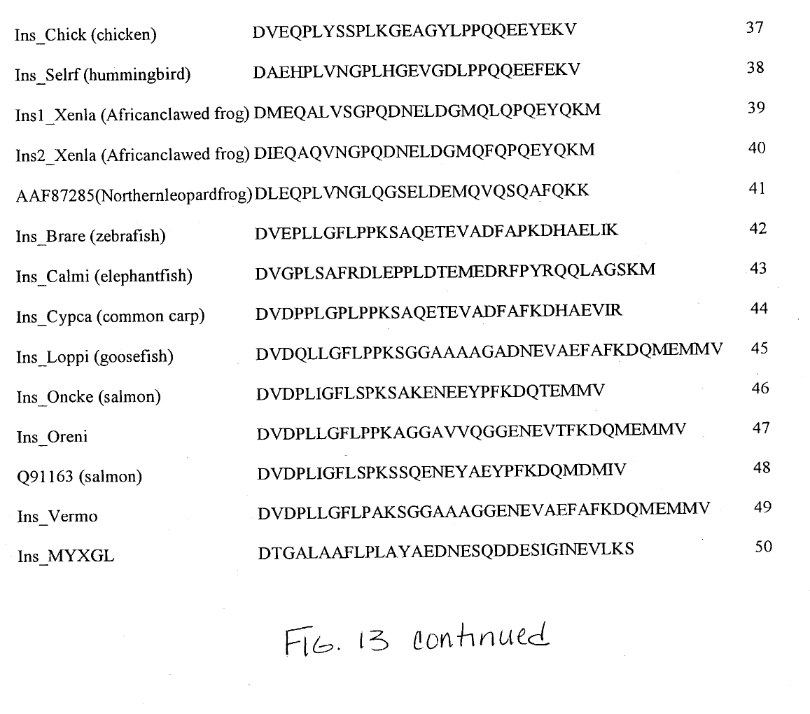

The term “insulin C-peptide” as used herein includes all forms of insulin C-peptide, including. native or synthetic peptides. Such insulin C-peptides may be human peptides, or may be from other animal species and genera, preferably mammals. C-peptides from a number of different species have been sequenced and are known in the art. It would thus be a routine matter to select a variant being a C-peptide from a species or genus other than human. Several such variants of C-peptide (i.e. representative C-peptides from other species) are shown in FIG. 12 (see SEQ ID NOS. 15-50). Thus variants and modifications of native insulin C-peptide are included as long as they retain insulin C-peptide activity. The insulin C-peptides may be expressed in their native form, ie. as different allelic variants as they appear in nature in different species or due to geographical variation etc., or as functionally equivalent variants or derivatives thereof, which may differ in their amino acid sequence, for example by truncation (e.g. from the N- or C-terminus or both) or other amino acid deletions, additions or substitutions. It is known in the art to modify the sequences of proteins or peptides, whilst retaining their useful activity and this may be achieved using techniques which are standard in the art and widely described in the literature e.g. random or site-directed mutagenesis, cleavage and ligation of nucleic acids etc.

Any such modifications, or combinations thereof may be made, as long as activity is retained. The C-terminal end of the molecule is believed to be important for activity. Preferably, therefore, the C-terminal end of the C-peptide should be preserved in any such C-peptide variants, more preferably the terminal pentapeptide of C-peptide should be preserved. Modifications to the mid-part of the C-peptide sequence (e.g., to residues 13 to 26 of human C-peptide) allow the production of functional variants of C-peptide and are hence covered.

Thus C-peptides may be used which have amino acid sequences which are substantially homologous, or substantially similar to the native C-peptide amino acid sequences for example to the human C-peptide sequence of SEQ ID NO. 1 or any of the other native C-peptide sequences shown in FIG. 13. Such substantially homologous sequences may include those having at least 30% (or more preferably at least 40, 50, 60, 70, 75, 80, 85, 90, 95, 98 or 99%) similarity to any one of SEQ ID Nos. 1 or 15 to 50 as shown in FIG. 13, preferably to the native human sequence of SEQ ID No. 1. Alternatively, the C-peptide may have an amino acid sequence having at least 30% (or more preferably at least 40, 50, 60, 70, 75, 80, 85, 90, 95, 98 or 99%) identity with the amino acid sequence of any one of SEQ ID Nos. 1 or 15 to 50 as shown in FIG. 13, preferably with the native human sequence of SEQ ID No. 1.

Amino acid sequence identity or similarity may be determined using the BestFit program of the Genetics Computer Group (GCG) Version 10 Software package from the University of Wisconsin. The program uses the local homology algorithm of Smith and Waterman with the default values: Gap creation penalty=8, Gap extension penalty=2, Average match=2.912, Average mismatch=2.003. Thus, functionally equivalent variants or derivatives of native insulin C-peptide sequences may readily be prepared according to techniques well known in the art, and include peptide sequences having a functional, e.g. a biological, activity of a native insulin C-peptide. Thus a variant of a naturally occurring wild-type or native C-peptide sequence may, for example, differ by 1 to 10, more preferably 1 to 6, or 1 to 4, or 1 to 3 amino acid substitutions, insertions and/or deletions which may be contiguous or non-contiguous as compared to the native or wild-type sequence (e.g. as compared to the sequence of any one of SEQ ID Nos. 1 or 15 to 50, preferably SEQ ID No. 1). Representative such variants may include those having 1 to 6, or more preferably 1 to 4, 1 to 3 or 1 or 2 amino acid substitutions as compared to SEQ ID No. 1. The substituted amino acid may be any amino acid, (e.g. any naturally occurring amino acid, particularly one of the well known 20 conventional amino acids (Ala(A); Cys(C); Asp(D); Glu(E); Phe(F); Gly(G); His(H);. Ile(I); Lys(K); Leu(L); Met(M); Asn(N); Pro(P); Gln(Q); Arg(R); Ser(S); Thr(T); Val(V); Trp(W); and Tyr(Y)). Conservative amino acid substitutions are preferred. As above, a proviso of such variants is that they retain C-peptide activity.

Thus, in terms of such activities, for example, insulin C-peptide is known to have an activity in stimulating Na⁺K⁺ATPase, which may underlie various of the therapeutic activities reported for C-peptide, e.g. in the treatment or diabetes or in the treatment or prevention of diabetic complications such as diabetic neuropathy, nephropathy and retinopathy. An assay for Na⁺K⁺ATPase activity is reported in WO 98/13384, incorporated herein by reference. Other activities of C-peptide have also been reported and the term “C-peptide activity” as used herein means any activity, exhibited by a native C-peptide, whether a physiological response exhibited in an in vivo or in vitro test system, or any biological activity or reaction mediated by a native C-peptide, for example in an enzyme assay or in binding to test tissues or membranes.

Thus, it is known that C-peptide increases the intracellular concentration of calcium. An assay for C-peptide activity can thus be by assaying for changes in intracellular calcium concentrations upon addition or administration of the peptide (e.g. fragment or derivative) in question. Such an assay is described in for example in Ohtomo et al., (1996), Diabetologia 39, 199-205; and Kunt et al., (1998), Diabetes 47, A30.

Further, C-peptide has been found to induce phosphorylation of the MAP-kinases ERK 1 and 2 of a mouse embryonic fibroblast cell line (Swiss 3T3), and measurement of such phosphorylation and MAPK activation may be used to assess, or assay for C-peptide activity, as described for example by Kitamura et al., (2001), Biochem. J. 355, 123-129.

C-peptide also has a well known effect in stimulating Na⁺K⁺ATPase activity and this also may form the basis of an assay for C-peptide activity, for example as described in WO 98/13384 or in Ohtomo et al., (1996), supra.

An assay for C-peptide activity based on endothelial nitric oxide synthase (eNOS) activity is also described in Kunt et al., supra, using bovine aortic cells and a reporter cell assay.

Finally, binding to particular cells may also be used to assess or assay for C-peptide activity, for example to cell membranes from human renal tubular cells, skin fibroblasts and saphenous vein endothelial cells using fluorescence correlation spectroscopy, as described for example in Rigler et al., (1999) PNAS USA 96, 13318-13323; Henriksson et al., (2000), Cell Mol. Life Sci. 57, 337-342; and Pramanik et al., (2001), BBRC 284, 94-98. Fragments of native or synthetic insulin C-peptide sequences may also have the desirable functional properties of the peptide from which they derive and are hence also included. Mention may be made in particular of the insulin C-peptide fragments described by Wahren et al., in WO98/13384. Such fragments include ELGGGPGAG, EGSLQ, ELGG, ELGGGP, GGPGA, GSLQ, GGGPGAG, GGGPG, GGGP, GGP, GGPG, LALEGSLQ, ALEGSLQ, LEGSLQ and fragments thereof. These fragments are capable of stimulating Na⁺K⁺ATPase to a similar or greater extent thatn C-peptide itself. All such analogues, variants, derivatives or fragments of insulin C-peptide are especially included in the scope of this invention, and are subsumed under the term “an insulin C-peptide”.

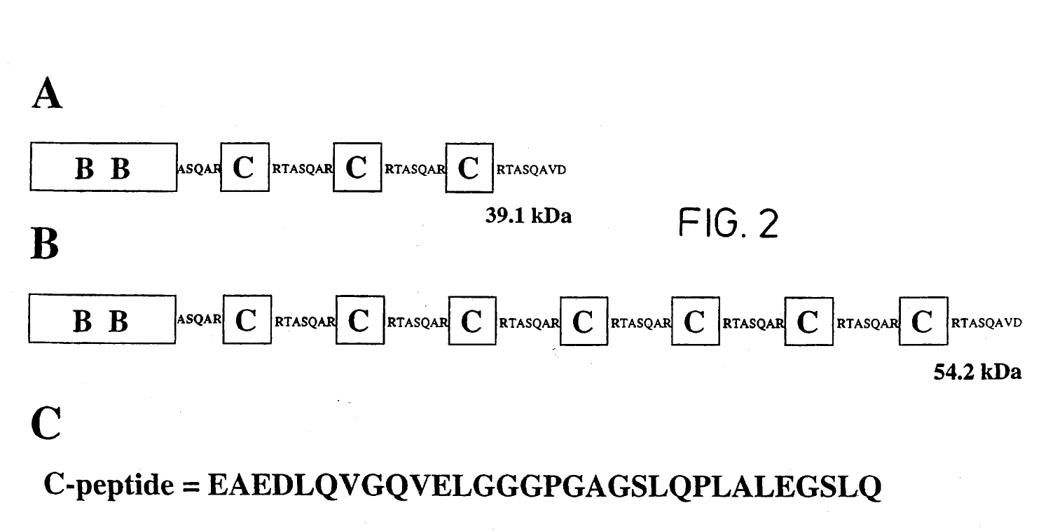

Conveniently, the native human insulin C-peptide may be used and is shown in FIG. 2C (SEQ ID. NO. 1.)

In a further preferred embodiment of the method according to the invention, the gene construct will additionally comprise a sequence which encodes a fusion partner (fusion tag) e.g. which is capable of binding to matrices used during processing of the product of gene expression.

The term “fusion partner” refers to any protein or peptide molecule or derivative or fragment thereof which is translated contiguously with the insulin C-peptide whose properties can be utilised in the further processing of the expressed fusion product.

The interaction between the fusion partner and the matrix may be based on affinity, chelating peptides, hydrophobic or charged interactions or any other mechanism known in the art. Conveniently, the fusion partner is one of a pair of affinity binding partners or ligands e.g. a protein, polypeptide or peptide sequence capable of selectively or specifically binding to or reacting with a ligand. Suitable fusion partners include for example streptococcal protein G and staphylococcal protein A and derivatives thereof, β-galactosidase, glutathione-S-transferase and avidin or streptavidin, or a fragment or derivative of any aforesaid protein, which have strong affinities with immunoglobulin G, substrate analogues or antibodies and biotin respectively. Such interactions can be utilised to purify the fused protein product from a complex mixture. The ZZ fragment of protein A (see Nilsson et al., supra) is an example of a protein fragment which may be used. Histidine peptides can be used as fusion partners as they bind to metal ions e.g. Zn²⁺, Cu²⁺ or Ni²⁺ and elution may be performed by lowering the pH or with EDTA (Ljungquist et al. (1989) Eur. J. Biochem. 186, 563-569). Particularly preferred polypeptide fusion partners are a 25 kDa serum albumin binding region (BB) derived from streptococcal protein G (SpG) (Nygren et al. (1988) J. Mol. Recogn. 1 69-74) or other SpG-derived albumin binding tags (Stahl and Nygren (1997) Path. Bio. 45, 66-76). Öberg et al., describe an expression vector, pTrp BB, (SEQ ID NO. 14) suitable for insertion of gene fragments for expression of a desired product as a fusion protein with BB (Proceedings of the 6th European Congress on Biotechnology, 1994, 179-182).

These fusion partners have a strong affinity to albumin and therefore purification of the expressed fusion protein can be based on ligand affinity chromatography e.g. using a column charged with albumin. The albumin is preferably immobilised on a solid support.

Any convenient means may be used to achieve the cleavage step, ie. the cleavage of the monomeric insulin C-peptides from the multimeric polypeptide i.e. from the expressed gene product, and optionally from the fusion partner if present. Conveniently, this may be achieved using enzymes. Preferably, the initial product of gene expression, i.e. the multimeric polypeptide or the fusion product or fusion protein, which comprises the fusion partner and multiple copies (monomers) of the insulin C-peptide, is cleaved by one or more proteolytic enzymes in a single process step to yield unfused single copies of the insulin C-peptide. A combined treatment with trypsin and carboxypeptidase B (e.g. from bovine, porcine or other sources) is a particularly preferred method of obtaining the desired cleavage products. Trypsin cleaves the proteins C-terminally of each arginine residue and carboxypeptidase B removes the C-terminal arginine present on each peptide after trypsin digestion. Conditions for achieving proteolytic cleavage are well known in the art, as are a range of other suitable proteolytic enzymes such as Subtilisin (including mutants thereof), Enterokinase, Factor Xa, Thrombin, IgA protease, Protease 3C, and Inteins. It has been found, for example, that incubation of the expressed gene product with the proteolytic enzymes (e.g. trypsin and carboxypeptidase B) for 60 minutes is sufficient for complete processing of the expressed protein. Conveniently, 5 minutes incubation time may be sufficient for adequate processing of the fusion protein such that no fusion or multimeric protein is detectable by conventional SDS PAGE. Alternatively, the initial product of gene expression may be cleaved by chemical reagents such as CNBr, hydroxylamine or formic acid.

Depending on the precise nature of the insulin C-peptide and nucleic acid molecule (genetic construct) used, the cleavage sites e.g. for proteolysis may be present naturally, or they may be introduced by appropriate manipulation of the genetic construct using known techniques e.g. site-directed mutagenesis, ligation of appropriate cleavage site-encoding nucleotide sequences etc.

Conveniently, the multimeric expressed polypeptide may include a linker region ie. a linker residue or peptide incorporating or providing a cleavage site. Advantageously, the cleavage site comprises a cleavable motif recognised and cleaved by a proteolytic enzyme. Linker regions may be incorporated between each “monomer” peptide in the multimeric construct, and/or optionally also between the fusion partner if present and a monomer peptide. Advantageously, each monomer peptide may be tandemly arranged with a linker region. Advantageously, the insulin C-peptide monomers in the multimer are flanked by appropriate linker sequences to ensure cleavage and release of insulin C-peptide free of any linker region residues. The linker region may comprise from 1 to 15 e.g. 1 to 12 or 1 to 10 amino residues, although the length is not critical and may be selected for convenience or according to choice. Linker regions of from 1 to 8, e.g. 1, 5 and 7 may be convenient. The individual linker region within each construct may be the same or different, although for convenience they are generally the same. Thus, for example, for cleavage by the combination of trypsin and carboxypeptidase B, linkers beginning or terminating in arginine residues may be provided.

An alternative linker may comprise the amino acid lysine, either solely or as part of a longer sequence and may also be cleaved by the trypsin/carboxypeptidase B combination.

For inclusion between insulin C-peptide monomers, such linkers may advantageously start with and terminate in such a cleavage site e.g. an arginine residue at both their N and C termini, to ensure release of an insulin C-peptide monomer without any additional amino acids. For inclusion between the fusion partner and/or at the end of the insulin C-peptide multimer, a single cleavage site (e.g. Arg) may be present at the appropriate terminus of the linker, (or correspondingly at an appropriate site for cleavage, depending on the precise linker sequence and cleavage enzymes used).

Exemplary representative linker regions include —RTASQAR— (SEQ ID NO. 2) for inclusion between C-peptide monomers, —ASQAR— (SEQ ID NO. 3) between the fusion partner and a C-peptide multimer and —RTASQAVD (SEQ ID NO. 4) at the end of the multimer.

As mentioned above, standard methods well-known in the art may be used for the introduction of linker sequences.

A further aspect of the present invention is a nucleic acid molecule comprising multiple copies of a nucleotide sequence encoding an insulin C-peptide, wherein said nucleic acid molecule encodes a multimeric polypeptide capable of being cleaved to yield single copies of said insulin C-peptide.

Alternatively viewed, this aspect of the invention can be seen to provide a nucleic acid molecule comprising a concatemer of a nucleotide sequence encoding an insulin C-peptide.

The various aspects of the invention set out above (and below) include embodiments where the multimeric polypeptide (gene product) does not include both an insulin A and an insulin B peptide, or where the nucleic acid molecule does not encode both an insulin A and B peptide. More particularly, in such embodiments, where the number of copies of insulin C-peptide in the multimeric polypeptide, or encoded by the nucleic acid molecule, is two, the multimeric polypeptide does not include, or the nucleic acid molecule does not encode, both insulin A and B peptides.

In a particularly preferred embodiment of the invention, the nucleic acid molecule will additionally comprise a nucleotide sequence which encodes a fusion partner which assists in the further processing of the encoded multimeric polypeptide e.g. which is useful for purification of the expressed protein product. The gene encoding the fusion partner will be in the correct position and orientation to be translated together with the multiple copies of the insulin C-peptide to form, initially, a single fused peptide. Suitable fusion proteins are discussed above.

Advantageously, the nucleic acid molecule will also comprise one or more nucleotide sequences encoding linker regions comprising cleavage sites, as discussed above.

As exemplary of nucleic acid molecules according to the invention may thus be mentioned those encoding a polypeptide of Formula (I)

H₂N-A-(C—X)ₙ—COOH (I)

wherein

C is an insulin C-peptide;

A is a bond, or a group F, wherein F is a fusion partner, or a group —(F—X)—;

X is a linker region comprising at least one cleavage site, each X being the same or different; and

n is an integer of 2 to 50.

This aspect of the invention includes an embodiment wherein Formula (I) includes the proviso that when n=2, said polypeptide (I) does not comprise an insulin A and B chain.

Insulin C-peptides (group C), fusion partners (group F) and linker regions (group X) may be as defined above. Likewise n may be as defined above in relation to the terms “multiple” and “multimeric”.

The nucleic acid molecule or genetic construct useful in the methods of the invention will preferably contain a suitable regulatory sequence which will control expression in the host cell. Such regulatory or expression control sequences include, for example, transcriptional (e.g. promoter-operator regions, ribosomal binding sites, termination stop sequences, enhancer elements etc.) and translational (e.g. start and stop codons) control elements, linked in matching reading frame to the coding sequences.

Any suitable host cell may be used, including prokaryotic and eukaryotic cells and may be selected according to the chosen expression system e.g. bacterial, yeast, insect (e.g. baculovirus-based) or mammalian expression systems. Very many different expression systems are known in the art and widely described in the literature. For example, E. coli can be used as host cells for peptide production, in which case, the regulating sequence may comprise, for example, the E. coli trp promoter. Other suitable hosts include Gram-negative bacteria other than E. coli, Gram-positive bacteria, yeast insect, plant or animal cells e.g. genetically engineered cell-lines.

Expression vectors which comprise the nucleic acid molecules described above constitute a further aspect of the present invention.

Any convenient vector may be used to achieve expression according to the methods of the invention and very many are known in the art and described in the literature. Suitable vectors thus include plasmids, cosmids or virus-based vectors. These vectors, which are introduced into the host cells for expression, are however, preferably plasmid, phage or virus vectors. The vectors may include appropriate control sequences linked in matching reading frame with the nucleic acid molecules of the invention. Other genetic elements e.g. replicons, or sequences assisting or facilitating transfer of the vector into the host cell, stabilising functions, e.g. to assist in maintenance of the vector in the host cell, cloning sites, restriction endonuclease cleavage sites or marker-encoding sequences may be included according to techniques well known in the art. The vectors may remain as discrete entities in the host cell or may, in the case of plasmid insertion vectors or other insertional vectors, be inserted into the host cell chromosome. Random non-specific integration into the host chromosome is possible, although specific homologous integration is preferred. Techniques for this are known in the art (see e.g. Pozzi et al. (1992) J. Res. Microbiol. 143, 449-457 and (1996) Gene 169, 85-90). The integration is “homologous” because the plasmid insertion vector comprises a segment of host cell chromosomal DNA.

Representative exemplary plasmids suitable for expressing genetic constructs, or nucleic acid molecules according to the invention include pTrpBB (Öberg et al., supra) or derivatives thereof. Alternatively such plasmids may be modified to remove sequences encoding the fusion partner if desired. Any high-copy number vector incorporating a Trp-promoter or similar may be used.

A variety of techniques are well known in the art and may be used to introduce such vectors into prokaryotic or eukaryotic cells for expression e.g. bacterial transformation techniques, transfection, electroporation. Transformed or transfected eukaryotic or prokaryotic host cells ie. host cells containing a nucleic acid molecule according to the invention and as defined above, form a further aspect of the invention.

As described in more detail in the Examples, expression vectors, specifically plasmids, harbouring the nucleic acid molecules of the invention have the advantage of genetic stability in their hosts; no genetic instability was detected in plasmids prepared from cultures grown to high cell densities, as assessed by restriction mapping.

A further aspect of the present invention provides a method for the production of a nucleic acid molecule which encodes a multimeric polypeptide comprising multiple copies of an insulin C-peptide, wherein the expressed multimeric polypeptide is capable of being subsequently cleaved to yield-single copies of the insulin C-peptide, said method comprising generating a nucleic acid molecule comprising multiple copies of a nucleotide sequence encoding an insulin C-peptide, linked in matching reading frame.

There are a number of techniques known in the art for generating multimeric copies of a gene or gene fragment which can be used in the methods of the present invention. For example, synthetic DNA fragments can be head-to-tail polymerised utilising designed single-stranded non-palindromic protruding ends. The polymerised DNA fragments can then be directly ligated to matching protrusions resulting from enzymatic restriction (Ljungquist et al. (1989) Eur. J. Biochem. 186, 563-569). Other methods to achieve multimerisation of gene fragments are based on the use of class IIS restriction enzymes such as Bsp MI (St\dot{a}hl et. al (1990) Gene 89, 87-193) or Bsm I (Haydn and Mandecki (1988) DNA 7, 571-577). Alternative strategies involve polymerisation of the gene construct and ligation of adapter molecules containing restriction sites to allow further subcloning (\dot{A}slund et al. (1987) Proc. Natl. Acad. Sci. USA 84, 1399-1403 and Irving et al. (1988) in Technological Advances in Vaccine Development, A. R. Liss Inc., New York 97-105). Methods for de novo synthesis of genes are also known, involving the use of the polymerase chain reaction (PCR), that would be suitable for the generation of multimeric gene fragments (Majumder (1992) Gene 110, 89-94) and Nguyen et al. (1994) in Advances in Biomagnetic Separation, Eaton Publishing Co., Natick 73-78).

In a preferred embodiment of the method according to the invention, the purified gene fragments (ie. nucleotide sequences encoding an insulin C-peptide) are allowed to polymerize in a head-to-tail fashion (multimerise), due to designed non-palindromic protrusions and are then ligated into a plasmid digested by a restriction enzyme, preferably Sfi I.

In a particularly preferred embodiment, a plasmid comprising a nucleotide sequence (e.g. a gene fragment) encoding an insulin C-peptide is digested to excise the said sequence or gene fragment and after multimerisation of the sequences or gene fragments they are ligated back into the digested plasmid. Transformants may advantageously be screened using a PCR-screening technique (St\dot{a}hl et al. (1993) Biotechniques 14, 424-434) which amplifies the segment encoding one or more copies of the insulin C-peptide. The PCR amplified fragments can be compared by agarose gel electrophoresis. In a further preferred embodiment, gene fragments encoding a desired number of concatamerized insulin C-peptides e.g. three or seven, are isolated and ligated into a further plasmid which has been digested using the same restriction enzyme as was used to excise the fragment encoding the insulin C-peptide. Most preferably, this later plasmid, which will be used for transformation of host cells, additionally comprises a suitable promoter and a sequence encoding a suitable fusion partner for the insulin C-peptide.

Further aspects of the invention include the products of the aforementioned methods, namely an insulin C-peptide multimer and the individual C-peptides released from said multimer by cleavage.

In particular, this aspect of the invention provides a multimeric polypeptide comprising multiple copies of an insulin C-peptide cleavable to release single copies of said insulin C-peptide. Optionally, the multimeric polypeptide may additionally comprise a fusion partner, and/or linker regions comprising a cleavage site flanking each said C-peptide monomer.

Also provided is a method for producing a multimeric polypeptide comprising multiple copies of an insulin C-peptide cleavable to release single copies of said insulin C-peptide, said method comprising culturing a host cell containing a nucleic acid molecule encoding said multimeric polypeptide under conditions whereby said multimeric polypeptide is expressed, and recovering the expressed multimeric polypeptide.

The host cells may be cultured using techniques known in the art e.g. batch or continuous culture formats.

The multimeric gene product or polypeptide may be recovered from the host cell culture using standard techniques well known in the art, e.g. standard cell lysis, and protein purification techniques. As mentioned above, where a fusion partner is included in the multimeric polypeptide, purification may readily be achieved based on affinity binding of the fusion partner.

A variety of techniques are known in the art for isolating proteins or polypeptides from cells or cell culture medium, both native and recombinantly expressed, and any of these may be used. Cell lysis to release intracellular proteins/polypeptides may be performed using any of the many methods known in the art and described in the literature, and if necessary further purification steps may be performed, again based on techniques known in the art, depending on whether batch or continuous culture methods are used.

Heat treatment methods for the lysis of cells and recovery of polypeptides have been found to be particularly effective in the case of the insulin C-peptide multimeric polypeptides of the present invention, for example the method described in WO90/00200 and modifications thereof. Such methods involve heating the host cell-containing culture medium e.g. for 50-100° C. for a period of time, generally not exceeding 1 hour, whereby the expressed polypeptide is released into the medium, advantageously in substantially pure form. This is believed to result from a selective release of the expressed polypeptide. In particular, it has surprisingly been observed that such a method works well in the case of soluble polypeptide products which are stable to the heat treatment, whether recombinant or not (and the method may thus be of more general applicability), but especially in the case of the insulin C-peptide multimeric polypeptide of the invention, where surprisingly high yields of high purity product may be obtained. Then, for example, such heat treatment may take place by heating at 80-100° C. e.g. 85-99° C. or 90-95° C. for 5-20 minutes, e.g. 8-10 minutes, and cooling thereafter, e.g. to 0-4° C. or on ice.

Following recovery of the multimeric polypeptide, it may be cleaved to release the individual insulin C-peptide monomers. Accordingly a further aspect of the invention provides a method for producing an insulin C-peptide, said method comprising cleaving a multimeric polypeptide as defined above, to release single copies of said insulin C-peptide.

Following cleavage of the multimeric polypeptide as discussed above to yield individual C-peptide monomers, these may also further be purified, e.g. to homogeneity (e.g. as demonstrated by SDS-PAGE) using well known standard techniques of purification e.g. ultrafiltration, size-exclusion chromatography, clarification, reversed-phase chromatography etc.

A further aspect of the present invention is the use in therapy of the cleaved peptide products of the methods described above. The cleaved insulin C-peptide can be used in the treatment of type 1 diabetes and/or diabetic complications. Also within the scope of the present invention therefore, is a method of treating type 1 diabetes or the complications thereof comprising administration of insulin C-peptide prepared by any of the methods described above.

The invention will now be described in more detail by way of non-limiting Examples and with reference to the following figures in which:

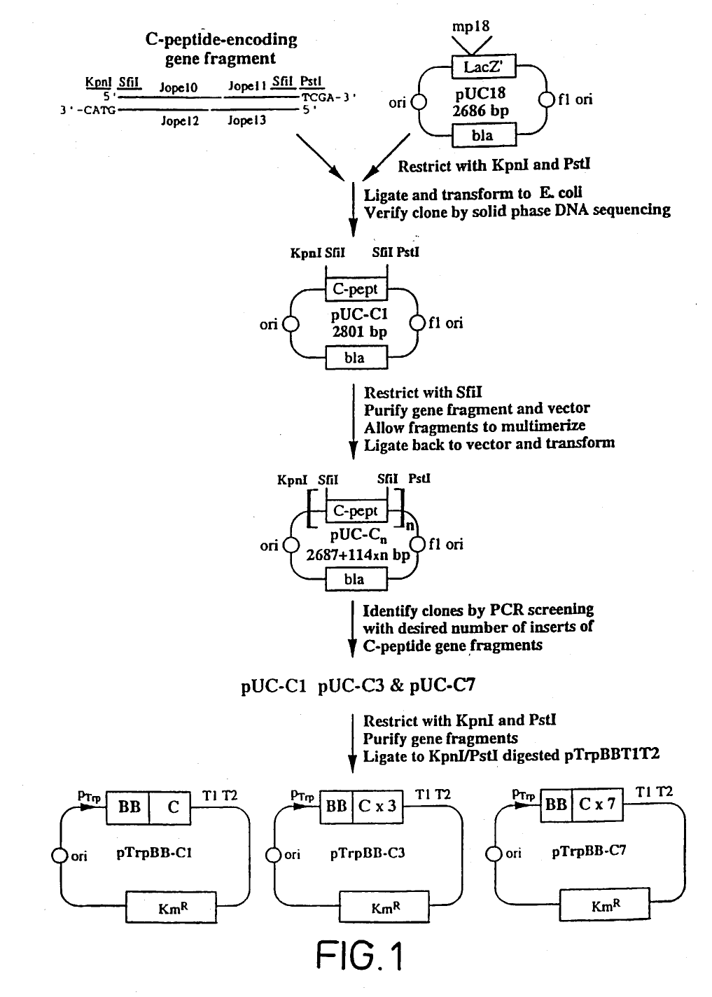

FIG. 1—is a schematic description of the production of gene constructs according to the invention, including the multimerization of the C-peptide-encoding gene fragment.

FIGS. 2A and B—are schematic descriptions of the two gene products, BB-C3(A) and BB-C7(B), with the linker regions flanking the C-peptide indicated in single letter code. Arginine residues (in bold) flank each C-peptide.

FIG. 2C—shows the amino acid sequence of the C-peptide in single letter code (SEQ ID. NO. 1).

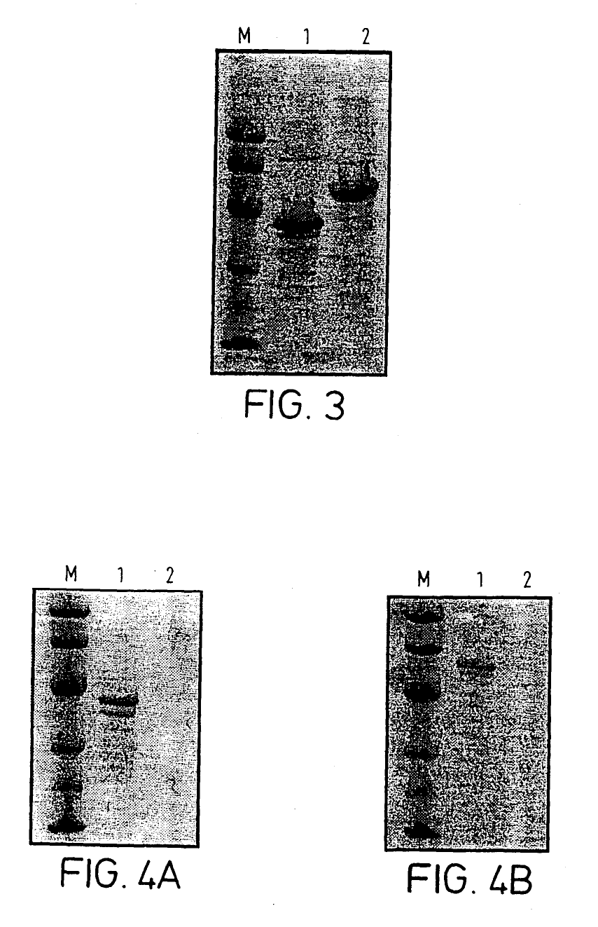

FIG. 3—is a copy of a photograph of an SDS-PAGE (10-15%) gel under reducing conditions of the two fusion proteins BB-C3 (Lane 1) and BB-C7 (Lane 2), respectively, after affinity purification on HSA-Sepharose. Marker proteins with molecular masses of 94, 67, 43, 30, 20 and 14 kDa, respectively appear in Lane M.

FIG. 4A—is a copy of a photograph of a SDS-PAGE (10-15%) gel under reducing conditions of BB-C3 and after incubation for various times with trypsin and carboxypeptidase B. Lane 1 shows the undigested fusion proteins and lane 2 protein digests after 5 minutes processing with trypsin and carboxypeptidase B. Lane M shows maker proteins with molecular masses of 94, 67, 43, 30, 20 and 14 kDa, respectively.

FIG. 4B is as for FIG. 4A, except the fusion product BB-C7 was examined here.

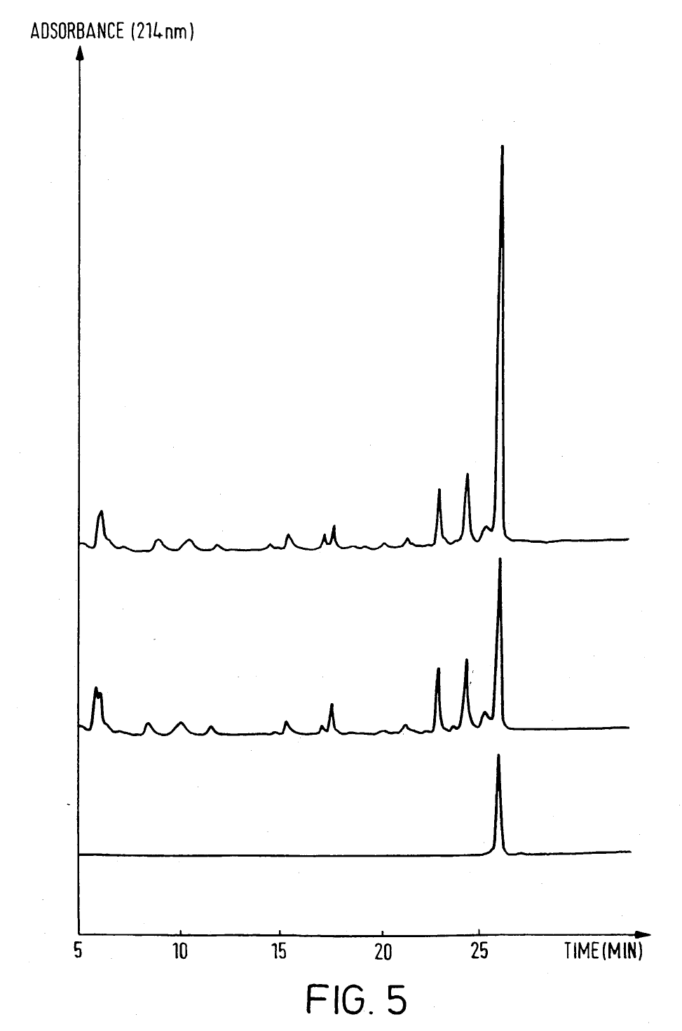

FIG. 5—shows reverse phase chromatography (RPC) analysis of the trypsin and carboxypeptidase B cleavage mixtures from equimolar amounts BB-C7 (upper) and BB-C3 (middle), respectively. Insulin C-peptide from Sigma (lower) was analysed as a control.

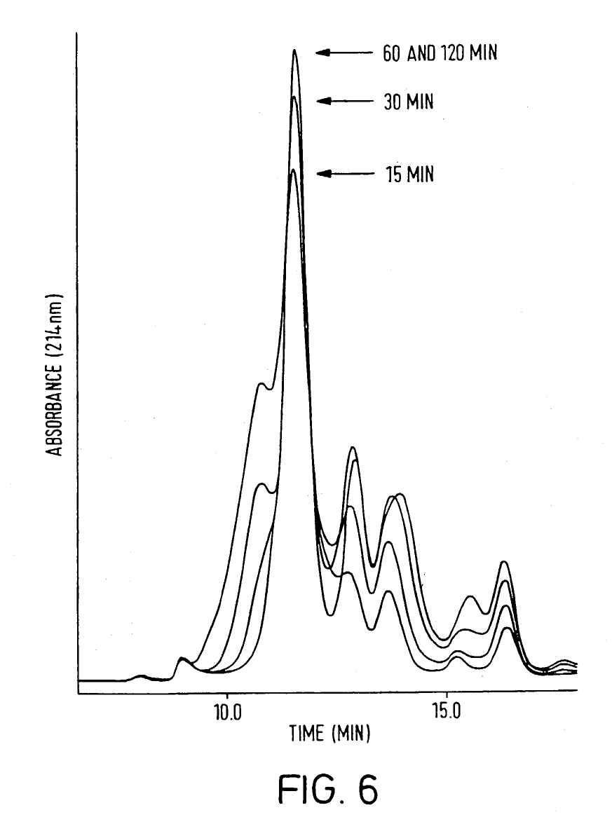

FIG. 6—shows overlay plots of size exclusion chromatograms (Superdex Peptide, Pharmacia Biotech, Uppsala, Sweden) of the BB-C7 fusion product processed for various times with trypsin (mass ratio 5000:1) and carboxypeptidase B (mass ratio 2000:1).

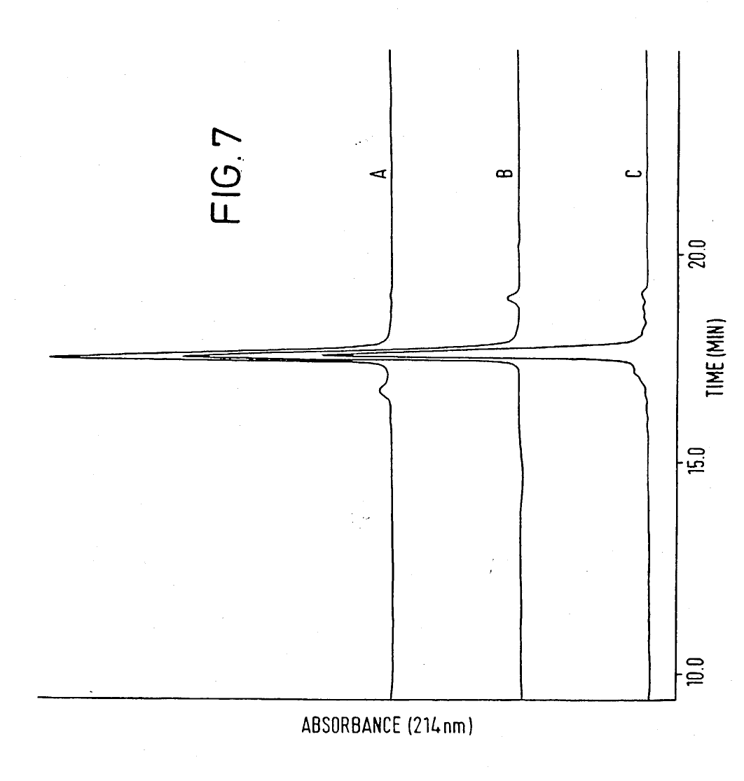

FIG. 7—shows reverse phase chromatography analysis of the insulin C-peptide originating from processed BB-C7(A) by comparison to insulin C-peptide standards provided by Eli Lilly (B) or purchased from Sigma (C).

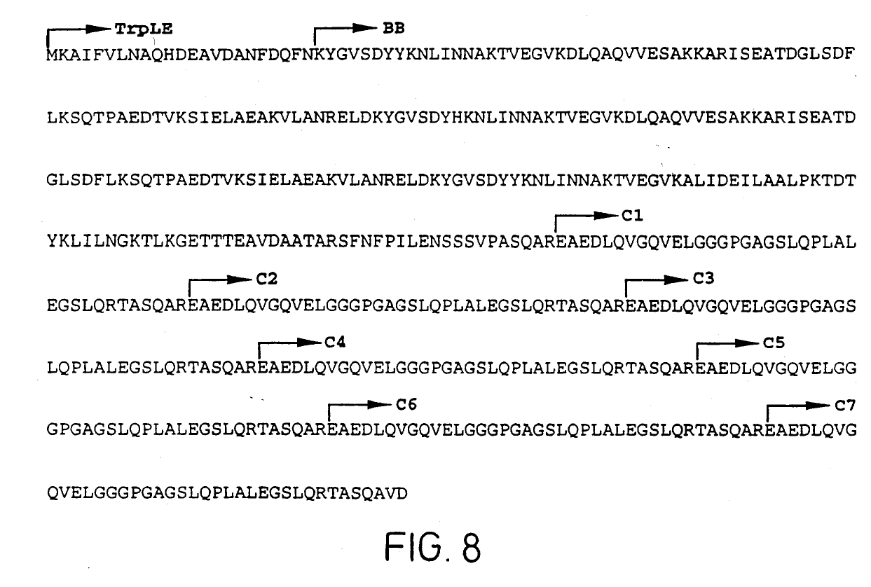

FIG. 8—illustrates the amino acid sequence in single letter code of the peptide product comprising the fusion partner BB and seven copies of the insulin C-peptide (SEQ ID NO. 5).

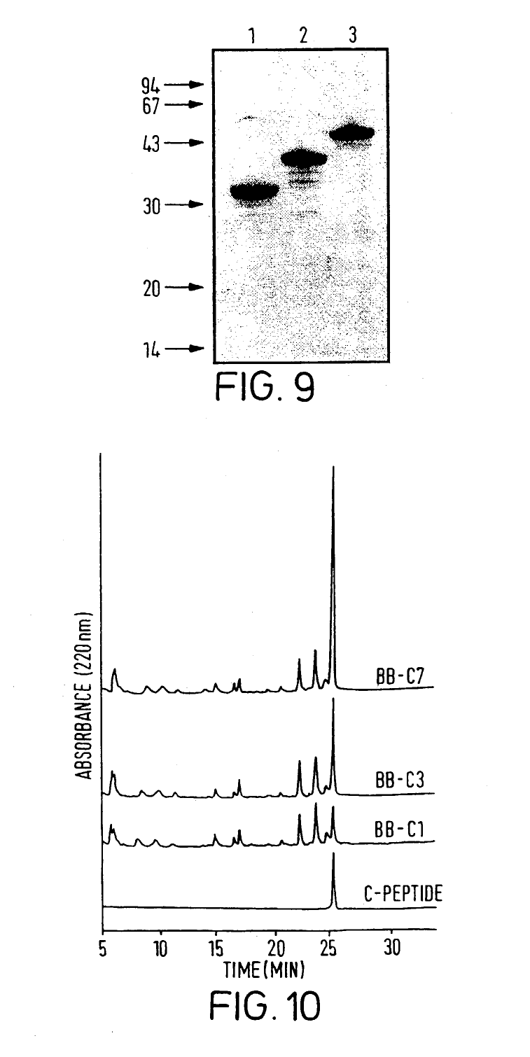

FIG. 9—shows analysis by SDS-10-15% PAGE of the synthesized fusion proteins, BB-Cl (lane 1), BB-C3 (lane 2) and BB-C7 (lane 3), after affinity purification on HSA-Sepharose. Molecular masses are to be indicated in kDa.

FIG. 10—shows RPC analysis of the trypsin +carboxypeptidase B cleavage mixtures from equimolar amounts of BB-C1, BB-C3 and BB-C7, respectively. A commercially available C-peptide standard (Sigma) was included as a control (see bottom).

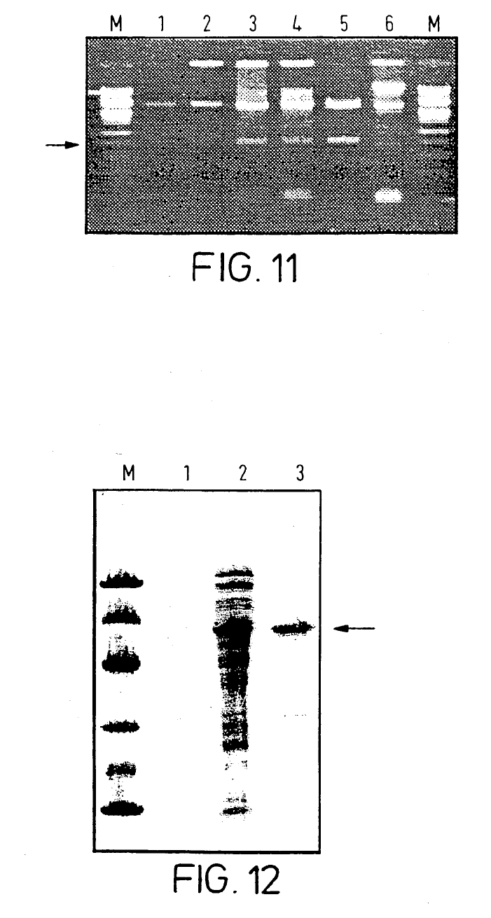

FIG. 11—shows agarose gel (1%) electrophoresis analysis of KpnI-PstI restriction of pTrpBB-C7 plasmid preparations from E. coli cultivations grown for 0 (Lane 1), 7 (Lane 2), 27 (Lane 3) or 31 hours (Lane 4). Lane 5 shows a KpnI-PstI restriction the original pTrpBB-C7 plasmid used for the initial transformation of the E. coli cells and lane 6 uncleaved pTrpBB-C7 after 31 hours of cultivation . The marker (M) lanes contains PstI-restricted lambda phage DNA. The arrow indicates the position for the C7 fragment.

FIG. 12—shows SDS-PAGE analysis (under reducing conditions) of samples from a BB-C7 cultivation. Lane 1: 2 μl of medium from an untreated culture. Lane 2: 0.5 μl of sonicated culture. Lane 3: 0.5 μl of medium after heat treatment of the culture. The arrow indicates the position of the BB-C7 fusion protein. Lane M shows marker proteins of molecular masses of 94, 67, 43, 30, 20 and 14 kDa.

FIG. 13—shows sequence alignment between C-peptide amino acid sequences from different species (SEQ ID NOS 1 and 15-50).

### EXAMPLE 1

Preparation of DNA Constructs

The four synthetic oligonucleotides Jope 10(5′-CGGCCTCCCA GGCCCGCGAA GCTGAGGACC TGCAAGTTGG TCAGGTTGAA CTGGGCGGTG GCCCGGGTGC AGGC-3′) (SEQ ID NO. 6), Jope 11 (5′-TCTTTGCAGC CGCTGGCTTT AGAAGGTTCT CTTCAGCGTA CGGCCTCCCA GGCCGTCGAC TAACTGCA-3′) (SEQ ID NO. 7), Jope 12 (3′-CATGGCCGGA GGGTCCGGGC GCTTCGACTC CTGGACGTTC AACCAGTCCA ACTTGACCCG CCACCGGG-5′) (SEQ ID NO. 8) and Jope 13 (3′-CCCACGTCCG AGAAACGTCG GCGACCGAAA TCTTCCAAGA GAAGTCGCAT GCCGGAGGGT CCGGCAGCTG ATTG-5′) (SEQ ID NO. 9) were phosphorylated and allowed to anneal pair-wise (Jope 10:Jope 12 and Jope 11:Jope 13) by incubation at 70° C. for 10 min with subsequent cooling to room temperature. The two created linkers were mixed and ligated to KpnI-PstI digested plasmid pUC18 (Yanish-Perron et al., 1985, Gene 33, 103-106) (FIG. 1), and the ligation mixture were transformed to the dcm-Escherichia coli strain GM31 (Marinus, (1973) Mol. Gen. Menet. 127, 47-55). A transformant (PUC-C1) with the correct nucleotide sequence in the inserted insulin C-peptide-encoding gene fragment was identified using PCR-based solid phase DNA sequencing (Hultman et al., (1989) Nucl. Acids Res. 17, 4937-4946). Plasmid DNA from pUC-C1 was prepared and after restriction with SfiI, both the excized insulin C-peptide-encoding gene fragment and the vector part were purified using the Mermaid-kit (glass-milk) (BIO 101 Inc., CA, USA) or the GeneClean-kit (BIO 101 Inc., CA, USA), respectively.

The purified insulin C-peptide gene fragments were allowed to polymerize in a head-to-tail fashion, due to designed non-palindromic protrusions, and were thereafter ligated back to the purified SfiI-digested plasmid. E. coli RRIΔM15 cells (Rüther, (1982) Nucl. Acids Res. 10, 5765-5772) were transformed with the ligation mixture and resulting transformants was screened using a PCR-screening technique (St\dot{a}hl et al., (1993) supra). Briefly, single colonies were picked to PCR tubes containing 50 μl PCR reaction mixture (20 mM TAPS, pH 9.3 at 20° C., 2 mM MgCl₂, 50 mM KCl, 0.1% Tween-20, 0.2 mM dNTP, 6 pmole of each primer (RIT27: 5′-GCTTCCGGCTCGTATGTGTG-3′ (SEQ ID NO. 10) and RIT28: 5′-AAAGGGGGATGTGCTGCAAG GCG-3′) (SEQ ID NO. 11) and 1.0 unit of Taq polymerase). The two PCR primers RIT27 and RIT28 have annealing sites in pUC18 flanking the insertion point for the insulin C-peptide fragments.

The PCR amplified fragments from clones with different number of inserted oligonucleotides were compared, with pUC18 as a reference, by agarose gel electrophoresis and transformants could be identified carrying one to seven inserts. The resulting plasmids were thus denoted pUC-C1, pUC-C2 etc.

Plasmids were prepared and gene fragments containing the desired number of inserts were excized by KpnI-PstI digestion. Gene fragments encoding one, three or seven concatamerized insulin C-peptides, respectively, were isolated and ligated to similarly digested pTrpBBT1T2, and the resulting plasmids were denoted pTrpBB-C1, pTrpBB-C3 and pTRpBB-C7, respectively. Plasmid pTrpBBT1T2 was constructed from plasmid pTrpBB (Öberg et al., (1994) in Proc. 6th Eur. Congress Biotechnol; Elsevier Science B. V. 179-182) by insertion of a transcription terminator sequence derived from plasmid pKK223-3 (Pharmacia Biotech, Uppsala, Sweden). The transcription terminator sequence was obtained from pKK223-3 using a standard PCR amplification protocol (Hultman et al., (1989) supra) and the oligonucleotides HEAN-19,5′-CCCCCTGCAGCTCGAGCGCCTTTA ACCTGTTTTGGCGGATG-3′ (SEQ ID NO. 12) and HEAN-20, 5′ CCCCAAGCTTAGAGTTTGTAG AAACGC-3′ (SEQ ID NO. 13).

The restriction sites introduced by PCR were digested with PstI and HindIII, followed by insertion into pTrpBB, previously digested with the same enzymes. The resulting expression vector pTrpBBT1T2 encodes an affinity handle consisting of a trp operon-derived leader sequences (eight amino acids) and a serum albumin binding region BB (25 kDa) (Nygren et al., (1988) supra) derived from streptococcal protein G. Transcription is under control of the E. coli trp promoter. In addition, the plasmid carries the gene for kanamycin resistance.

### EXAMPLE 2

Protein Expression and Purification

E. coli cells harbouring pTrpBB-C3 and pTrpBB-C7, and thus encoding the fusion proteins BB-C3 and BB-C7, respectively, were grown overnight at 37° C. in shake-flasks containing 10 ml Tryptic Soy Broth (Difco, USA) (30 g/l) supplemented with yeast extract (Difco) (5 g/l) and kanamycin monosulfate (50 mg/l). The overnight cultures were diluted 10-fold to 100 ml into baffled shake-flasks having the same type of media and grown at 37° C. Gene expression was induced at mid-log phase (A_{600 nm}≅1) by the addition of β-indole acrylic acid to 25 mg/l. Cells were harvested 20 hours after induction, by centrifugation at approximately 6000 g for 10 min. Cells were resuspended in 1/20 of the culture volume in TST (50 mM Tris-HCl pH 8.0. 200 mM NaCl, 0.05% Tween 20, 1 mM EDTA), lysed by sonication and centrifuged at approximately 40,000 g. The samples for the sonication were prepared by sedimenting the shake-flask culture by centrifugation, and thereafter resuspending the cells in 30 ml of cold TST buffer. The samples were stored on ice during a 2 minute pulsed sonication which was performed on a Sonics and Materials Inc. (Danbury, Conn., USA) Vibra Cell (500 W) using a 13 mm standard horn tip, a 70% duty cycle (20 kHz) and with the output control set to 6.5. The supernatants, containing soluble cytoplasmic proteins, were filtered (0.45 μm) and diluted to 100 ml with TST. The soluble fusion proteins were isolated by affinity chromatography on human-serum-albumin (HSA)-Sepharose (Nygren et al., (1988) supra) as described by Stahl et al (1989) J. Immunol. Meth. 124, 43-52. Eluted fractions were monitored for protein content by absorbance measurement at 280 nm and relevant fractions were lyophilised.

FIG. 3 shows affinity purified BB-C3 and BB-C7, respectively, after a single step purification on HSA-Sepharose. Full-length products were predominant for both fusion proteins which also migrated in accordance with their molecular masses; 39.1 and 54.2 kDa, respectively. The expression levels for shake-flask cultivations were almost identical for the two fusion proteins; being 130 mg/l for BB-C3 and 120 mg/l for BB-C7.

### EXAMPLE 3

Proteolytic Digestion of the Fusion Proteins

Trypsin, which cleaves C-terminally of basic amino acid residues, has been used for a long time to cleave fusion proteins. Despite expected low specificity, trypsin has been shown to be useful for specific cleavage of fusion proteins, leaving basic residues within folded protein domains uncleaved (Wang et al., (1989) J. Biol. Chem. 264, 21116-21121). Trypsin has the additional advantages of being inexpensive and readily available.

Here we have used trypsin in combination with carboxypeptidase B for the processing of BB-C3 and BB-C7, respectively, in order to obtain native human insulin C-peptide. Trypsin would thus cleave the fusion proteins C-terminally of each arginine residue and carboxypeptidase B would remove the C-terminal arginine present on each insulin C-peptide monomer after trypsin digestion.

To analyze the efficiency of the processing, the two fusion proteins, BB-C3 and BB-C7 were incubated with trypsin and carboxypeptidase B for various times and subjected to SDS/PAGE analysis. It was found that both fusion proteins were processed rapidly and after 5 minutes processing, no fusion protein could be visualized by the SDS/PAGE analysis (FIGS. 4A and B).

In addition, an analysis was performed to compare the relative yields of insulin C-peptide monomers after cleavage of the fusion proteins BB-C3 and BB-C7, respectively. The cleavage mixtures after trypsin and carboxypeptidase B treatment of equimolar amounts of BB-C3 and BB-C7, respectively, were analysed by reverse phase HPLC (250 mm, Kromasil C8 column, 4.6 mm inner diameter, particle size 7 μm, Hewlett Packard 1090). Elution was performed using a 10-40% acetonitrile gradient containing 0.1% trifluoroacetic acid during 30 minutes at 40° C. As can be seen in FIG. 5, a significantly higher ratio between the insulin C-peptide product (elution time ca. 25.4 min) and other cleavage products (of BB fusion partner origin) was obtained from cleavage of the BB-C7 fusion protein compared to cleavage of the BB-C3 fusion protein. Integration of the insulin C-peptide peak areas (C7:C3) gave a peak area ratio of 2.43, close to the theoretical 2.33.

This does not give any information about when the fusion proteins are completely processed. To investigate when the trypsin-carboxypeptidase B treatment has reached completion, the fusion protein, BB-C7 was subjected to enzymatic processing for various times. The lyophilized BB-C7 fusion protein was dissolved in 100 mM phosphate buffer, pH 7.5, containing 0.1% (by vol.) Tween 20 to a protein concentration of 1 mg/ml, respectively, Trypsin (T-2395, Sigma, St. Louis, Mo., USA) and carboxypeptidase B (Boehringer Mannheim) were added to trypsin/fusion protein ratios of 1/5000 (by mass) and carboxypeptidase B/fusion protein ratios of 1/2000 (by mass), respectively. After 15, 30, 60 and 120 minutes, samples were taken from the cleavage mixtures and the digestions were stopped by decreasing the pH to 3 by adding HAc. Acetonitrile to 20% (by vol.) was added in order to stabilize the cleavage products.

The cleavage material was analyzed by size-exclusion chromatography (Superdex Peptide column on SMART™ system, Pharmacia Biotech, Uppsala, Sweden) and by making overlay plots of the chromatograms (FIG. 6), it could be concluded that BB-C7 was completely processed after 60 minutes under these conditions since no additional insulin C-peptide was obtained by increased incubation times. These results also indicate that it would be possible to obtain quantitative yields of insulin C-peptide from fusion proteins comprising multimeric forms of insulin C-peptide.

### EXAMPLE 4

Characterization of the Obtained Insulin C-peptide: Reversed Phase Chromatography (RPC) and Mass Spectrometry

In order to confirm that the obtained peptide really corresponds to native human insulin C-peptide, two different analyses were performed. Firstly, reversed phase chromatography (RPC) analysis was used for comparison of RPC-purified insulin C-peptide obtained by processing of BB-C7 to insulin C-peptide standards, said standards being C-peptide obtained from Eli Lilly (CA, USA) and commercially available insulin C-peptide fragment 3-33 (Sigma, USA). The insulin C-peptide preparations were analyzed by RPC on a Sephasil C8 5 μm SC2. 1/10 column using the SMART™ system (Pharmacia Biotech, Uppsala, Sweden). Elution was performed using a gradient of 26-36% acetonitrile containing 0.1% (by vol.) trifluoroacetic acid during 20 min at 25° C. The flow rate was 100 μl/min and the absorbance was measured at 214 nm. It could be concluded that all three preparations were close to identical, having the same retention time and the same low level of impurities (FIG. 7). Secondly, the insulin C-peptide obtained from BB-C7 was subjected to mass spectrometry (Table 1). The protein mass determination was performed using a JEOL SX102 mass spectrometer (JEOL, Japan)) equipped with an electrospray unit. The good agreement in mass (Table 1), together with the observed similarities to insulin C-peptide standards in the comparative RPC analysis, suggest that native human insulin C-peptide was obtained.

TABLE 1
Molecular mass of insulin C-peptide (Da)

| | |
|---|---|
| Calculated | 3020.3 |
| Experimental | 3019.7 ± 1.8 |

### EXAMPLE 5

Characterization of the Obtained Insulin C-peptide Monomer: Radioimmunoassay (RIA)

The insulin C-peptide monomer obtained from cleavage of the fusion protein BB-C7 was analyzed using a commercially available radioimmunoassay developed to monitor human insulin C-peptide levels in e.g. blood and urine (Euro-Diagnostica, Malmö, Sweden; cat. no. MD 315). For comparison, also a preparation of insulin C-peptide (Eli-Lilly Co, Indianapolis, Ind., USA), previously demonstrated to be biologically active (Johansson et al., (1992) Diabetologia 35:1151-1158), was analyzed. Samples for analysis were prepared by weighing followed by dilution to final concentrations of 3.31 and 3.30 nanomoles/litre of the two preparations of C-peptide, respectively, in 0.05 M Na-phosphate buffer, pH 7.4, 5% human albumin serum (HSA) and 0.02% Thimerosal. Briefly, the assay involves a rabbit anti-human C-peptide antiserum, ¹²⁵-human insulin C-peptide tracer, a goat anti-rabbit Ig antiserum-PEG reagent, human insulin C-peptide standards and control samples for quantification of insulin C-peptide in assayed samples after the construction of a standard curve. The results from the analysis of the two samples are summarized in Table 2 below. The results show that the two preparations are equally recognized and quantified using the RIA assay.

TABLE 2
Comparative RIA analysis of insulin C-peptide
with demonstrated biological activity and insulin
C-peptide obtained from cleavage of the recombinant
fusion protein BB-C7.

| | Expected concentration | Assayed concentration |
|---|---|---|
| Sample | (nM) | (nM) |
| Insulin C-peptide | 3.31 | 2.34 (71%) |
| (from cleavage | | |
| of fusion protein | | |
| BB-C7) | | |
| Insulin C-peptide | 3.30 | 2.41 (73%) |
| (from Eli-Lilly) | | |

### EXAMPLE 6

Expression, Purification and Proteolytic Digestion of Fusion Proteins BB-C1, BB-C3 and BB-C7

This Example presents additional comparative results regarding the BB-C1 fusion protein, for the experiments presented in Examples 2 and 3.

E. coli cells harbouring plasmids pTrp BB-C1, pTrp BB-C3 or pTrp BB-C7 respectively (see Example 1) were grown, and the fusion proteins were expressed, obtained, purified and analysed as described in Example 2.

Analysis of E. coli cells transformed with either pTrp BB-C1, pTrp BB-C3 or pTrp BB-C7 showed that the encoded fusion proteins, BB-C1, BB-C3 and BB-C7 accumulated intracellularly as soluble gene products (data not shown). After cell disruption, the produced fusion proteins were efficiently purified by HSA-affinity chromatography. FIG. 9 shows the affinity purified BB-C1, BB-C3 and BB-C7 fusion proteins, respectively, after a single step purification on HSA-Sepharose. Full-length products were predominant for the three fusion proteins, which also migrated in accordance to their molecular masses; 31.5, 39.1 and 54.2 kDa, respectively.

The expression levels for shake-flask cultures were reproducible and similar for the three different fusion proteins, in the range of 40-60 mg/l.

To analyse the efficiency of the processing of the three affinity-purified fusion proteins, BB-C1, BB-C3 and BB-C7 were incubated with trypsin and carboxypeptidase B for various times and subjected to SDS-PAGE analysis. The three fusion proteins were processed rapidly, and after 5 minutes of treatment, no remaining full-length fusion protein was detected by the SDS-PAGE (data not shown).

Efficiency of proteolytic processing was further analysed as described in Example 3, and it was found that BB-C7 was completely cleaved after 60 minutes.

In order to compare more adequately the relative yields of C-peptide monomers after cleavage of the BB-C1, BB-C3 and BB-C7 fusion proteins, respectively, a reverse phase HPLC analysis was performed (as described in Example 3).

The cleavage mixtures from a 120 minute trypsin+carboxypeptidase B treatment of approximately equimolar amounts of BB-C1, BB-C3 and BB-C7, respectively, were analysed. (The A₂₂₀ₙₘ was monitored). Results (FIG. 10) demonstrated a significantly higher ratio between the C-peptide product and other cleavage products of the BB-C7 and BB-C3 fusion proteins, as compared to cleavage of the BB-C1 fusion protein. Approximately equimolar amounts of each fusion protein were loaded on the RPC column, as demonstrated by the equal peak heights originating from trypsin-digested BB-tag visible in the three chromatograms (FIG. 10). Integration of the C-peptide peak areas (940, 2324 and 5647 absorbance units×s×10⁻³ for BB-C1, BB-C3 and BB-C7 respectively) resulted in ratios of 2.5 for C3:C1 and 6.0 for C7:C1, being close to the theoretical values 3 and 7, respectively.

The results further show an improved yield of insulin C-peptide monomers from insulin C-peptide multimers (C3, C7) as compared with a monomeric fusion protein (C1).

### EXAMPLE 7

Investigation of Genetic Stability for the Plasmid pTrpBB-C7 Encoding the BB-C7 Fusion Protein

This example describes how the genetic stability for the plasmid pTrpBB-C7 encoding the BB-C7 fusion protein was assessed. E. coli cells harbouring plasmid pTrpBB-C7 were grown for different times and samples were taken after 0, 7, 27 and 31 hours of cultivation. Thirty-one hours would resemble a cultivation time for a large-scale fermentation production of BB-C7. Plasmids were recovered from the samples according to standard protocols (Sambrook et al., A Laboratory Manual, Second Edition, Cold Spring Harbor Laboratory Press, New York).

The plasmids were subjected to KpnI-PstI restriction, in order to excize the fragment encoding the C7 concatamer (see FIG. 1). The original pTrpBB-C7 plasmid used for the initial transformation of the E. coli cells was included as control, and was thus also subjected to KpnI-PstI restriction. As can be seen in FIG. 11, the restricted fragment has the same size from all samples, verifying that the plasmid pTrpBB-C7 would be genetically stable during cultivations for extended times.

### EXAMPLE 8

Heat Treatment for Selective Release of BB-C7 into the Culture Medium

This example describes how the BB-C7 fusion protein could be released into the culture medium by heat treatment and thereby significantly improve the purity of the starting material for further purification of BB-C7. Background: Compared to the most widely used method for releasing recombinant proteins produced intracellularly in E. coli (including the unit operations centrifugation, resuspension of the cell pellet in a appropriate buffer and cell disruption by high pressure homogenisation), the release of the gene product by the heat treatment method have many advantages: (i) a production scheme including the heat treatment have one clarification step less, (ii) the stability of the gene product increases due to heat denaturation of host cell proteases, (iii) a significant initial purification of the gene product is obtained by the precipitation of other E. coli proteins and, (iv) the release of nucleic acids is reduced compared to a total disruption of the cells. The method would be suitable also for release of other intracellularly expressed recombinant proteins that are soluble also at high expression levels and that are stable to the heat treatment required to release the protein.

E. coli cells harbouring plasmid pTrpBB-C7, encoding BB-C7, were cultivated as described in Example 2. As an alternative to the described sonication process (Example 2) for cell disrupture, a heat treatment step could be utilized for a selective and efficient release of BB-C7 into the culture medium. The culture was at the end of the cultivation submerged into a water bath with boiling water for 8-10 minutes. The culture had after this time reached a temperature of approximately 90° C. The shake-flask was thereafter placed on ice. As can be seen in FIG. 12 (lane 3), at this temperature, the BB-C7 fusion protein is released into the culture medium without release of substantial amounts of host proteins.

The host proteins are most likely completely denatured by this treatment. In contrast, sonication (FIG. 12, lane 2) and other mechanical methods for cell disrupture would release also all host proteins as well as nucleic acids, resulting in a very heteregenous starting material for further purification of BB-C7. Very little protein is normally secreted by the E. coli culture (FIG. 12, lane 1). The BB-C7 was found to be stable to the heat treatment and could be further purified and processed for release of C-peptide monomers as described in Examples 1-3.

1. A method of producing an insulin C-peptide, which comprises expressing in a host cell a multimeric polypeptide comprising multiple copies of a said insulin C-peptide, and cleaving said expressed polypeptide to release single copies of the insulin C-peptide.

2. A nucleic acid molecule comprising multiple copies of a nucleotide sequence encoding an insulin C-peptide, wherein said nucleic acid molecule encodes a multimeric polypeptide capable of being cleaved to yield single copies of said insulin C-peptide.

3. A method for the production of a nucleic acid molecule which encodes a multimeric polypeptide comprising multiple copies of an insulin C-peptide, wherein the expressed multimeric polypeptide is capable of being subsequently cleaved to yield single copies of the insulin C-peptide, said process comprising generating a nucleic acid molecule comprising multiple copies of a nucleotide sequence encoding an insulin C-peptide, linked in matching reading frame.

4. A multimeric polypeptide comprising multiple copies of an insulin C-peptide, wherein said multimeric polypeptide can be cleaved to release single copies of said insulin C-peptide.

5. A method of producing a multimeric polypeptide which contains multiple copies of an insulin C-peptide and can be cleaved to release single copies of said insulin C-peptide, said method comprising culturing a host cell containing a nucleic acid molecule encoding said multimeric polypeptide under conditions whereby said multimeric polypeptide is expressed, and recovering the expressed multimeric polypeptide.

6. A method of producing an insulin C-peptide, said method comprising cleaving a multimeric polypeptide as defined in claim 4.

7. A method according to claim 1, wherein said multiple copies of said insulin C-peptide are arranged in tandem.

8. A method according to claim 1, wherein said multimeric polypeptide comprises 2 to 30 copies of said insulin C-peptide.

9. A method according to claim 8, wherein said multimeric polypeptide comprises 3 to 7 copies of said insulin C-peptide.

10. A method according to claim 1, wherein said multimeric polypeptide further comprises a fusion partner.

11. A method according to claim 10, wherein said fusion partner is one of a pair of affinity binding partners or ligands.

12. A method according to claim 11, wherein said fusion partner is the 25 kDa serum albumin binding region (BB) derived from streptococcal protein G.

13. A method according to claim 1, wherein the insulin C-peptide monomers in said multimeric polypeptide are flanked by linker regions comprising a cleavage site.

14. A method according to claim 13, wherein said cleavage site is cleavable by a proteolytic enzyme.

15. A method according to claim 14, wherein said cleavage site comprises arginine residues for cleavage by trypsin and carboxypeptidase B.

16. An expression vector comprising a nucleic acid molecule as defined in claim 2.

17. The expression vector according to claim 16, said expression vector being a plasmid.

18. The expression vector according to claim 17, wherein said expression vector is based on plasmid pTrpBB (SEQ ID NO: 14).

19. A host cell containing a nucleic acid molecule as defined in claim 2.

20. An insulin C-peptide produced by the method of claim 1.

21. An insulin C-peptide produced by the method of claim 6.

22. The nucleic acid molecule according to claim 2, wherein said multiple copies of said insulin C-peptide or said insulin C-peptide encoding-nucleotide sequence are arranged in tandem.

23. The nucleic acid molecule according to claim 2, wherein said multimeric polypeptide comprises 2 to 30 copies of said insulin C-peptide.

24. The nucleic acid molecule according to claim 23, wherein said multimeric polypeptide comprises 3 to 7 copies of said insulin C-peptide.

25. The nucleic acid molecule according to claim 2, wherein said multimeric polypeptide further comprises a fusion partner.

26. The nucleic acid molecule according to claim 25, wherein said fusion partner is an affinity binding partner or a ligand.

27. The nucleic acid molecule according to claim 26, wherein said fusion partner is a 25 kDa serum albumin binding region (BB) derived from streptococcal protein G.

28. The nucleic acid molecule according to claim 2, wherein each insulin C-peptide in said multimeric polypeptide is flanked by linker regions comprising a cleavage site.

29. A nucleic acid molecule according to claim 28, wherein said cleavage site is cleavable by a proteolytic enzyme.

30. A nucleic acid molecule according to claim 29, wherein said cleavage site comprises arginine residues for cleavage by trypsin and carboxypeptidase B.

31. A nucleic acid molecule according to claim 2, wherein said nucleic acid molecule further comprises one or more regulatory or expression control sequences.

32. The nucleic acid molecule according to claim 2, wherein said multiple copies of said nucleotide sequence encoding said insulin C-peptide are in matching reading frame.

33. The multimeric polypeptide according to claim 4, wherein said multiple copies of said insulin C-peptide are arranged in tandem.

34. The multimeric polypeptide according to claim 4, wherein said multimeric polypeptide comprises 2 to 30 copies of said insulin C-peptide.

35. The multimeric polypeptide according to claim 34, wherein said multimeric polypeptide comprises 3 to 7 copies of said insulin C-peptide.

36. The multimeric polypeptide according to claim 4, wherein said multimeric polypeptide further comprises a fusion partner.

37. The multimeric polypeptide according to claim 36, wherein said fusion partner is an affinity binding partner or a ligand.

38. The multimeric polypeptide according to claim 37, wherein said fusion partner is a 25 kDa serum albumin binding region (BB) derived from streptococcal protein G.

39. The multimeric polypeptide according to claim 4, wherein each insulin C-peptide in said multimeric polypeptide is flanked by linker regions comprising a cleavage site.

40. The multimeric polypeptide according to claim 39, wherein said cleavage site is cleavable by a proteolytic enzyme.

41. The multimeric polypeptide according to claim 40, wherein said cleavage site comprises arginine residues for cleavage by trypsin and carboxypeptidase B.

42. The method according to claim 5, wherein said multiple copies of said insulin C-peptide are arranged in tandem.

43. The method according to claim 5, wherein said multimeric polypeptide comprises 2 to 30 copies of said insulin C-peptide.

44. The method according to claim 43, wherein said multimeric polypeptide comprises 3 to 7 copies of said insulin C-peptide.

45. The method according to claim 5, wherein said multimeric polypeptide further comprises a fusion partner.

46. The method according to claim 45, wherein said fusion partner is an affinity binding partner or a ligand.

47. The method according to claim 46, wherein said fusion partner is a 25 kDa serum albumin binding region (BB) derived from streptococcal protein G.

48. The method according to claim 5, wherein each insulin C-peptide in said multimeric polypeptide is flanked by linker regions comprising a cleavage site.

49. The method according to claim 48, wherein said cleavage site is cleavable by a proteolytic enzyme.

50. The method according to claim 49, wherein said cleavage site comprises arginine residues for cleavage by trypsin and carboxypeptidase B.


SEQUENCE LISTING (63 sequences)

SEQ ID NO: 1 - protein, 31 aa, Homo sapiens
EAEDLQVGQVELGGGPGAGSLQPLALEGSLQ

SEQ ID NO: 2 - protein, 7 aa, Artificial Sequence
  Representative linker region
RTASQAR

SEQ ID NO: 3 - protein, 5 aa, Artificial Sequence
  Representative linker region
ASQAR

SEQ ID NO: 4 - protein, 8 aa, Artificial Sequence
  Representative linker region
RTASQAVD

SEQ ID NO: 5 - protein, 521 aa, Artificial Sequence
  Synthetically generated polypeptide
MKAIFVLNAQHDEAVDANFDQFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATARSFNFPILENSSSVPASQAREAEDLQVGQVELGGGPGAGSLQPLALEGSLQRTASQAREAEDLQVGQVELGGGPGAGSLQPLALEGSLQRTASQAREAEDLQVGQVELGGGPGAGSLQPLALEGSLQRTASQAREAEDLQVGQVELGGGPGAGSLQPLALEGSLQRTASQAREAEDLQVGQVELGGGPGAGSLQPLALEGSLQRTASQAREAEDLQVGQVELGGGPGAGSLQPLALEGSLQRTASQAREAEDLQVGQVELGGGPGAGSLQPLALEGSLQRTASQAVD

SEQ ID NO: 6 - DNA, 74 bp, Artificial Sequence
  Oligonucleotide for PCR
cggcctcccaggcccgcgaagctgaggacctgcaagttggtcaggttgaactgggcggtggcccgggtgcaggc

SEQ ID NO: 7 - DNA, 68 bp, Artificial Sequence
  Oligonucleotide for PCR
tctttgcagccgctggctttagaaggttctcttcagcgtacggcctcccaggccgtcgactaactgca

SEQ ID NO: 8 - DNA, 68 bp, Artificial Sequence
  Oligonucleotide for PCR
gggccaccgcccagttcaacctgaccaacttgcaggtcctcagcttcgcgggcctgggaggccggtac

SEQ ID NO: 9 - DNA, 74 bp, Artificial Sequence
  Oligonucleotide for PCR
gttagtcgacggcctgggaggccgtacgctgaagagaaccttctaaagccagcggctgcaaagagcctgcaccc

SEQ ID NO: 10 - DNA, 20 bp, Artificial Sequence
  Oligonucleotide for PCR
gcttccggctcgtatgtgtg

SEQ ID NO: 11 - DNA, 23 bp, Artificial Sequence
  Oligonucleotide for PCR
aaagggggatgtgctgcaaggcg

SEQ ID NO: 12 - DNA, 41 bp, Artificial Sequence
  Oligonucleotide for PCR
ccccctgcagctcgagcgcctttaacctgttttggcggatg

SEQ ID NO: 13 - DNA, 27 bp, Artificial Sequence
  Oligonucleotide for PCR
ccccaagcttagagtttgtagaaacgc

SEQ ID NO: 14 - DNA, 4646 bp, Artificial Sequence
  Expression vector
gcggccgctaattcatgctgtggtgtcatggtcggtgatcgccagggtgccgacgcgcatctcgactgcacggtgcaccaatgcttctggcgtcaggcagccaatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcgaactagttaactagtacgcaagttcacgtaaaaagggtatctagaattatgaaagcaattttcgtactgaatgcgcaacacgatgaagccgtagacgcgaatttcgaccaattcaacaaatatggagtaagtgactattacaagaatctaatcaacaatgccaaaactgttgaaggcgtaaaagaccttcaagcacaagttgttgaatcagcgaagaaagcgcgtatttcagaagcaacagatggcttatctgatttcttgaaatcacaaacacctgctgaagatactgttaaatcaattgaattagctgaagctaaagtcttagctaacagagaacttgacaaatatggagtaagtgactatcacaagaacctaatcaacaatgccaaaactgttgaaggtgtaaaagaccttcaagcacaagttgttgaatcagcgaagaaagcgcgtatttcagaagcaacagatggcttatctgatttcttgaaatcacaaacacctgctgaagatactgttaaatcaattgaattagctgaagctaaagtcttagctaacagagaacttgacaaatatggagtaagtgactattacaagaacctaatcaacaatgccaaaactgttgaaggtgtaaaagcactgatagatgaaattttagctgcattacctaagactgacacttacaaattaatccttaatggtaaaacattgaaaggcgaaacaactactgaagctgttgatgctgctactgcaagatctttcaatttccctatcctcgagaattcgagctcggtaccggcctcccaggcccgcgaagctgaggacctgcaagttggtcaggttgaactgggcggtggcccgggtgcaggctctttgcagccgctggctttagaaggttctcttcagcgtacggcctcccaggcccgcgaagctgaggacctgcaagttggtcaggttgaactgggcggtggcccgggtgcaggctctttgcagccgctggctttagaaggttctcttcagcgtacggcctcccaggcccgcgaagctgaggacctgcaagttggtcaggttgaactgggcggtggcccgggtgcaggctctttgcagccgctggctttagaaggttctcttcagcgtacggcctcccaggcccgcgaagctgaggacctgcaagttggtcaggttgaactgggcggtggcccgggtgcaggctctttgcagccgctggctttagaaggttctcttcagcgtacggcctcccaggcccgcgaagctgaggacctgcaagttggtcaggttgaactgggcggtggcccgggtgcaggctctttgcagccgctggctttagaaggttctcttcagcgtacggcctcccaggcccgcgaagctgaggacctgcaagttggtcaggttgaactgggcggtggcccgggtgcaggctctttgcagccgctggctttagaaggttctcttcagcgtacggcctcccaggcccgcgaagctgaggacctgcaagttggtcaggttgaactgggcggtggcccgggtgcaggctctttgcagccgctggctttagaaggttctcttcagcgtacggcctcccaggccgtcgactaactgcagctcgagcgcttaactgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctaagctttggtgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccggatcccggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgaacgcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatta

SEQ ID NO: 15 - protein, 31 aa, Pan troglodytes; identical to SEQ ID NO: 1

SEQ ID NO: 16 - protein, 29 aa, Aotus trivirgatus
EAEDLQVGQVELGGGSITGSLPPLEGPMQ

SEQ ID NO: 17 - protein, 31 aa, Crabeatingmacaque
EAEDPQVGQVELGGGPGAGSLQPLALEGSLQ

SEQ ID NO: 18 - protein, 31 aa, African green monkey polyomavirus; identical to SEQ ID NO: 17

SEQ ID NO: 19 - protein, 29 aa, Sus scrofa
EAENPQAGAVELGGGLGGLQALALEGPPQ

SEQ ID NO: 20 - protein, 26 aa, Bos taurus
EVEGPQVGALELAGGPGAGGLEGPPQ

SEQ ID NO: 21 - protein, 31 aa, Equus caballus
EABDPQVGEVELGGGPGLGGLQPLALAGPQQ

SEQ ID NO: 22 - protein, 26 aa, Ovis aries
EVEGPQVGALELAGGPGAGGLEGPPQ

SEQ ID NO: 23 - protein, 31 aa, Canis familiaris
EVEDLQVRDVELAGAPGEGGLQPLALEGALQ

SEQ ID NO: 24 - protein, 31 aa, Oryctolagus cuniculus
EVEELQVGQAELGGGPGAGGLQPSALELALQ

SEQ ID NO: 25 - protein, 31 aa, Rattus norvegicus
EVEDPQYPQLELGGGPEAGDLQTLALEVARQ

SEQ ID NO: 26 - protein, 31 aa, Rattus norvegicus
EVEDPQVAQLELGGGPGAGDLQTLALEVARQ

SEQ ID NO: 27 - protein, 31 aa, Rodentia sp.
EVEDPQVGQVELGAGPGAGSEQTLALEVARQ

SEQ ID NO: 28 - protein, 29 aa, Mus musculus
EVEDPQVEQLELGGSPGDLQTLALEVARQ

SEQ ID NO: 29 - protein, 31 aa, Mus musculus
EVEDPQVAQLELGGGPGAGDLQTLALEVAQQ

SEQ ID NO: 30 - protein, 31 aa, Cavia porcellus
ELEDPQYEQTELGMGLGAGGLQPLALEMALQ

SEQ ID NO: 31 - protein, 31 aa, Cricetulus longicaudatus
GYEDPQVAQLELGGGPGADDLQTLALEVAQQ

SEQ ID NO: 32 - protein, 31 aa, Psammomys obesus
GYDDPQMPQLELGGSPGAGDLRALALEVARQ

SEQ ID NO: 33 - protein, 29 aa, Ocide
ELEDLQVEQAELGLEAGGLQPSALEMILQ

SEQ ID NO: 34 - protein, 18 aa, Mus musculus
GGPGAGDLQTLALEVAQQ

SEQ ID NO: 35 - protein, 16 aa, Mus musculus
GSPGDLQTLALEVARQ

SEQ ID NO: 36 - protein, 28 aa, Anas platyrhynchos
  VARIANT (1)...(28): Xaa = Any Amino Acid
DVEQPLVNGPLKGEVGELPPQHEEYQXX

SEQ ID NO: 37 - protein, 28 aa, Gallus gallus
DVEQPLYSSPLKGEAGYLPPQQEEYEKV

SEQ ID NO: 38 - protein, 28 aa, Selasphorus rufus
DAEHPLVNGPLHGEVGDLPPQQEEFEKV

SEQ ID NO: 39 - protein, 28 aa, Xenopus laevis
DMEQALVSGPQDNELDGMQLQPQEYQKM

SEQ ID NO: 40 - protein, 28 aa, Xenopus laevis
DIEQAQVNGPQDNELDGMQFQPQEYQKM

SEQ ID NO: 41 - protein, 28 aa, Rana pipiens
DLEQPLVNGLQGSELDEMQVQSQAFQKK

SEQ ID NO: 42 - protein, 32 aa, Danio rerio
DVEPLLGFLPPKSAQETEVADFAPKDHAELIK

SEQ ID NO: 43 - protein, 34 aa, Callorhinchus milii
DVGPLSAFRDLEPPLDTEMEDRFPYRQQLAGSKM

SEQ ID NO: 44 - protein, 32 aa, Cyprinus carpio
DVDPPLGPLPPKSAQETEVADFAFKDHAEVIR

SEQ ID NO: 45 - protein, 38 aa, Lophius piscatorius
DVDQLLGFLPPKSGGAAAAGADNEVAEFAFKDQMEMMV

SEQ ID NO: 46 - protein, 30 aa, Oncorhynchus keta
DVDPLIGFLSPKSAKENEEYPFKDQTEMMV

SEQ ID NO: 47 - protein, 34 aa, Oreochromis niloticus
DVDPLLGFLPPKAGGAWQGGENEVTFKDQMEMMV

SEQ ID NO: 48 - protein, 32 aa, Lepeophtheirus salmonis
DVDPLIGFLSPKSSQENEYAEYPFKDQMDMIV

SEQ ID NO: 49 - protein, 37 aa, Verasper moseri
DVDPLLGFLPAKSGGAAAGGENEVAEFAFKDQMEMMV

SEQ ID NO: 50 - protein, 33 aa, Myxine glutinosa
DTGALAAFLPLAYAEDNESQDDESIGINEVLKS

SEQ ID NO: 51 - protein, 9 aa, Artificial Sequence
  Fragment
ELGGGPGAG

SEQ ID NO: 52 - protein, 5 aa, Artificial Sequence
  Fragment
EGSLQ

SEQ ID NO: 53 - protein, 4 aa, Artificial Sequence
  Fragment
ELGG

SEQ ID NO: 54 - protein, 6 aa, Artificial Sequence
  Fragment
ELGGGP

SEQ ID NO: 55 - protein, 5 aa, Artificial Sequence
  Fragment
GGPGA

SEQ ID NO: 56 - protein, 4 aa, Artificial Sequence
  Fragment
GSLQ

SEQ ID NO: 57 - protein, 7 aa, Artificial Sequence
  Fragment
GGGPGAG

SEQ ID NO: 58 - protein, 5 aa, Artificial Sequence
  Fragment
GGGPG

SEQ ID NO: 59 - protein, 4 aa, Artificial Sequence
  Fragment
GGGP

SEQ ID NO: 60 - protein, 4 aa, Artificial Sequence
  Fragment
GGPG

SEQ ID NO: 61 - protein, 8 aa, Artificial Sequence
  Fragment
LALEGSLQ

SEQ ID NO: 62 - protein, 7 aa, Artificial Sequence
  Fragment
ALEGSLQ

SEQ ID NO: 63 - protein, 6 aa, Artificial Sequence
  Fragment
LEGSLQ